(19) United States
(12) Patent Application Publication
(10) Pub. No.: US 2004/0228819 A1
(43) Pub. Date: Nov. 18, 2004

(54) Cosmetic system for application as a multi-step cosmetic product

(75) Inventors: Thomas Elliot Rabe, Baltimore, MD (US); Crystal Ann Turner, Baltimore, MD (US); Randy Sean Moreland, Bel Air, MD (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/840,834

(22) Filed: May 7, 2004

Related U.S. Application Data
(60) Provisional application No. 60/471,244, filed on May 16, 2003.

(51) Int. Cl. A61K 7/021

(52) U.S. Cl. 424/63

(57) ABSTRACT

A cosmetic system suitable for application to the skin as a multi-step cosmetic product. The system includes two compositions. The first composition is composed of from about 5 to about 90% of a low-volatility material with a vapor pressure of less than 10 mmHg at 1 atm and 25° C. and from about 0.05 to about 10% of an emulsifier which is soluble or dispersible in the low-volatility material. The second composition is composed of a continuous phase fluid, solid, gel, or semi-solid having an interfacial tension with the low-volatility material from the first composition which is at least 0 dyne/cm. The second composition is topically applied to the skin after the first composition. The invention further relates to methods of using the cosmetic system in order to effectively deliver a satisfactory foundation product to a user's skin.

### CROSS REFERENCE TO RELATED APPLICATION(S)

This application claims the benefit of U.S. Provisional Application No. 60/471,244, filed May 16, 2003.

### FIELD OF INVENTION

The present invention relates to a cosmetic system that is suitable for use as a multi-step cosmetic product. In particular, the cosmetic system includes at least two compositions that are topically and sequentially applied to skin.

### BACKGROUND

It is well known in the skin beauty care field that cosmetic compositions, such as foundation, can be used to provide skin benefits including shine control, oil absorption, natural skin appearance, skin feel, and transfer resistance. However, these skin benefits are not simultaneously achieved through one foundation system because of the challenge of formulating the compositions to meet multiple consumer needs. For example, a foundation with transfer resistance properties typically does not have good skin feel attributes. Further, a composition with superior oil absorption benefits may not have good transfer resistance properties.

A need still exists for a cosmetic composition with superior transfer resistance, shine control, application, skin feel, and appearance benefits to be delivered through one foundation system. Applicants have found that it is possible to dramatically improve these benefits utilizing a two step system which enables the consumer to apply higher levels of key benefit actives to the skin surface without the negative application, feel, or appearance properties that incorporating the same levels of actives into a single formulation would provide. Furthermore, in developing two step cosmetic systems, it has been found that it is important to design the cosmetically acceptable carriers for the two steps so that they work synergistically together in improving application, feel, and appearance properties. Therefore, a need still exists for a foundation with superior benefits delivered through a two-step foundation system wherein the spreading properties of the second step in the system are made superior by a specific formulation approach that allows the second step to be emulsified into the first step upon application.

### SUMMARY OF THE INVENTION

The present invention relates to a cosmetic system suitable for application to the skin as a multi-step cosmetic product. The system includes at least two compositions. The first composition is composed of from about 5% to about 90% of a low-volatility fluid, gel, or semi-solid with a vapor pressure of at least about 0 mmHg to about 10 mmHg at 1 atm and 25° C. and from 0.05-10% of an emulsifier which is soluble or dispersible in the low-volatility material. The second composition is composed of a continuous phase fluid, solid, gel, or semi-solid which is at least partially immiscible with the low-volatility material from the first composition. In the case where the two continuous phases are fluids, this can be quantified by the existence of a meniscus between the two phases and a measurable interfacial tension which greater than 0 dyne/cm. The second composition is topically applied to the skin after the first composition.

The invention further relates to methods of using the cosmetic system in order to effectively deliver a satisfactory foundation product to a user's skin.

### DETAILED DESCRIPTION OF THE INVENTION

The present invention relates to a cosmetic system suitable for application as a multi-step cosmetic product. Surprisingly, inventors of the present invention have found that it is possible to create a foundation with superior benefits though a two-step foundation system wherein the spreading properties of the second step in the system are made superior by a specific formulation approach that allows the second step to be emulsified into the first step upon application. The ability to emulsify the second step into the first step results in unexpected benefits in application/spreading properties as well as lubricious feel and natural appearance. By contrast, if the continuous phase of the second composition were in fact soluble/miscible with the film remaining from the first composition, then the second composition would actually re-solubilize what remains from the first composition and create a thicker, draggier, application experience. This problem is further compounded with the incorporation of film-forming polymers and particulates that must then be re-dissolved/re-wetted by the second composition.

The essential components of the cosmetic system are described below. Also included is a nonexclusive description of various optional and preferred components useful in embodiments of the present invention.

As used herein, “safe and effective amount” means an amount of a compound, component, or composition (as applicable) sufficient to significantly induce a positive effect (e.g., confer a noticeable cosmetic benefit), but low enough to avoid serious side effects, (e.g., undue toxicity or allergic reaction), i.e., to provide a reasonable benefit to risk ratio, within the scope of sound medical judgment.

As used herein, “cosmetic system” means any color cosmetic or skin care product. “Cosmetic systems” include, but are not limited to, products that leave color on the face, including make-up, liquid foundation, mascara, concealers, eye liners, brow colors, eye shadows, blushers, lip sticks, lip balms, face powders, solid emulsion foundations, powder foundations, and the like. The term “foundation” refers to liquid, creme, mousse, pancake, compact, concealer or like product created or reintroduced by cosmetic companies to even out the overall coloring of the skin. Additionally, “cosmetic systems” may include moisturizers, sunscreen products, self-tanning products, antiperspirant compositions, shaving creams, and skin cleansers.

Herein, “comprising” means that other steps and other ingredients which do not affect the end result can be added. This term encompasses the terms “consisting of” and “consisting essentially of”. The products, compositions, and methods/processes of the present invention can comprise, consist of, and consist essentially of the essential elements and limitations of the invention described herein, as well as any of the additional or optional ingredients, components, steps, or limitations described herein.

All percentages, parts, and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

The preferred pH range for the cosmetic composition is from about pH 3 to about pH 10, preferably from about pH 4 to about pH 9.

All measurements made are at 25° C., unless otherwise designated.

### FIRST COMPOSITION

A. A Low-Volatility Material

The cosmetic system of the present invention includes a low-volatility material. Suitable low-volatility materials for inclusion in the claimed cosmetic systems include, but are not limited to hydrophobic non-polar materials, hydrophilic polar materials, and mixtures thereof. Low-volatility fluids, gels and semi-solids of the present invention are defined as having a vapor pressure of at least about 0 mmHg to about 10 mm Hg at 1 atm and 25° C., preferably at least about 0 mmHg to about 5 mm Hg at 1 atm and 25° C., and even more preferably at least about 0 mmHg to about 3 mmHg at 1 atm and 25° C.

Hydrophobic non-polar materials of the present invention are defined as having a solubility parameter of approximately 11.0 or less as determined on the Hildebrand scale. Hydrophilic polar materials of the present invention are defined as having a solubility parameter of about 12.0 or greater as determined on the Hildebrand scale. Hildebrand developed a method for deriving the solubility parameter from the boiling point, molecular weight, and specific gravity of a material; J. H. Hildebrand, J. M. Prausnitz and R. L. Scott, Regular and Related Solutions, New York; Van Nostrand Reinholdt (1950), herein incorporated by reference. This Hildebrand solubility parameter is published for many cosmetic and pharmaceutical materials in the Cosmetic Bench Reference, Carol Stream Ill., Allured Publishing (1992) and in A. F. Barton, Handbook of Solubility Parameters and Other Cohesion Parameters, 2nd ed., Boca Raton; CRC Press (1992).

The present cosmetic systems comprise from about 5.0% to about 90.0%, by weight of the cosmetic system of the low-volatility materials, preferably from about 8.0% to about 70.0% by weight of the cosmetic system. In preferred embodiments, low-volatility materials are present in the cosmetic system in an amount of from about 10.0% to about 60.0%, by weight of the cosmetic system.

The first composition may also include more volatile materials with a vapor pressure of greater than about 10 mmHg at 1 atm and 25° C. in combination with the low volatility materials. Examples of suitable hydrophilic and hydrophobic fluids for the first composition may be selected from the following materials, as long as they adhere to the volatility criteria of the invention.

1. Hydrophobic Non-Polar Materials:

Nonlimiting examples of suitable hydrophobic components include those selected from the group consisting of:

(1) Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p. 415-417 (1993).

(2) Petrolatum, which is also known as petroleum jelly, is a colloidal system of nonstraight-chain solid hydrocarbons and high-boiling liquid hydrocarbons, in which most of the liquid hydrocarbons are held inside the micelles. See The Merck Index, Tenth Edition, Entry 7047, p. 1033 (1983); Schindler, Drug. Cosmet. Ind., 89, 36-37, 76, 78-80, 82 (1961); and International Cosmetic Ingredient Dictionary, Fifth Edition, vol. 1, p. 537 (1993).

(3) Straight and branched chain hydrocarbons having from about 7 to about 40 carbon atoms. Nonlimiting examples of these hydrocarbon materials include dodecane, isododecane, squalane, cholesterol, hydrogenated polyisobutylene, docosane (i.e. a C₂₂ hydrocarbon), hexadecane, isohexadecane (a commercially available hydrocarbon sold as Permethyl® 101A by Presperse). Also useful are the C7-C40 isoparaffins, which are C7-C40 branched hydrocarbons.

(4) C1-C30 alcohol esters of C1-C30 carboxylic acids and of C2-C30 dicarboxylic acids, including straight and branched chain materials as well as aromatic derivatives (as used herein in reference to the hydrophobic component, mono- and poly-carboxylic acids include straight chain, branched chain and aryl carboxylic acids). Nonlimiting examples include diisopropyl sebacate, diisopropyl adipate, isopropyl myristate, isopropyl palmitate, methyl palmitate, myristyl propionate, 2-ethylhexyl palmitate, isodecyl neopentanoate, di-2-ethylhexyl maleate, cetyl palmitate, myristyl myristate, stearyl stearate, isopropyl stearate, methyl stearate, cetyl stearate, behenyl behenrate, dioctyl maleate, dioctyl sebacate, diisopropyl adipate, cetyl octanoate, diisopropyl dilinoleate.

(5) mono-, di- and tri-glycerides of C1-C30 carboxylic acids, e.g., caprilic/capric triglyceride, PEG-6 caprylic/capric triglyceride, PEG-8 caprylic/capric triglyceride.

(6) alkylene glycol esters of C1-C30 carboxylic acids, e.g., ethylene glycol mono- and di-esters, and propylene glycol mono- and di-esters of C1-C30 carboxylic acids e.g., ethylene glycol distearate.

(7) propoxylated and ethoxylated derivatives of the foregoing materials.

(8) C1-C30 mono- and poly-esters of sugars and related materials. These esters are derived from a sugar or polyol moiety and one or more carboxylic acid moieties. Depending on the constituent acid and sugar, these esters can be in either liquid or solid form at room temperature. Examples of liquid esters include: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C18 mono-and/or di-unsaturated and behenic, in a molar ratio of unsaturates:behenic of 1:7 to 3:5. A particularly preferred solid sugar polyester is the octaester of sucrose in which there are about 7 behenic fatty acid moieties and about 1 oleic acid moiety in the molecule. Other materials include cottonseed oil or soybean oil fatty acid esters of sucrose. The ester materials are further described in, U.S. Pat. No. 2,831,854; U.S. Pat. No. 4,005,196; U.S. Pat. No. 4,005,195; U.S. Pat. No. 5,306,516; U.S. Pat. No. 5,306,515; U.S. Pat. No. 5,305,514; U.S. Pat. No. 4,797,300; U.S. Pat. No. 3,963,699; U.S. Pat. No. 4,518,772; and U.S. Pat. No. 4,517,360.

(9) Organopolysiloxane oils. Nonlimiting examples of suitable silicones are disclosed in U.S. Pat. No. 5,069,897. Examples of suitable organopolysiloxane oils include polyalkylsiloxanes, cyclic polyalkylsiloxanes, and polyalkylarylsiloxanes.

Polyalkylsiloxanes useful in the composition herein include polyalkylsiloxanes with viscosities of from about 0.5 to about 1,000,000 centistokes at 25° C. Such polyalkylsiloxanes can be represented by the general chemical formula R₃ SiO[R₂ SiO]ₓ SiR₃ wherein R is an alkyl group having from about 1 to about 30 carbon atoms (preferably R is methyl or ethyl, more preferably methyl; also mixed alkyl groups can be used in the same molecule), and x is an integer of from about 0 to about 10,000, chosen to achieve the desired molecular weight which can range to over about 10,000,000. Commercially available polyalkylsiloxanes include the polydimethylsiloxanes, which are also known as dimethicones, examples of which include the Vicasil® series sold by General Electric Company and the Dow Corning® series sold by Dow Corning Corporation. Specific examples of suitable polydimethylsiloxanes include Dow Corning® 200 fluid having a viscosity of 0.65 centistokes and a boiling point of 100° C., Dow Corning® 225 fluid having a viscosity of 10 centistokes and a boiling point greater than 200° C., and Dow Corning® 200 fluids having viscosities of 50, 350, and 12,500 centistokes, respectively, and boiling points greater than 200° C. Suitable dimethicones include those represented by the chemical formula (CH₃)₃ SiO[(CH₃)₂ SiO]ₓ [CH₃ RSiO]_{y} Si(CH₃)₃ wherein R is straight or branched chain alkyl having from about 2 to about 30 carbon atoms and x and y are each integers of 1 or greater selected to achieve the desired molecular weight which can range to over about 10,000,000. Examples of these alkyl-substituted dimethicones include cetyl dimethicone and lauryl dimethicone.

Cyclic polyalkylsiloxanes suitable for use in the composition include those represented by the chemical formula [SiR₂—O]ₙ wherein R is an alkyl group (preferably R is methyl or ethyl, more preferably methyl) and n is an integer from about 3 to about 8, more preferably n is an integer from about 3 to about 7, and even more preferably n is an integer from about 4 to about 6. When R is methyl, these materials are typically referred to as cyclomethicones. Commercially available cyclomethicones include Dow Corning® 244 fluid having a viscosity of 2.5 centistokes, and a boiling point of 172° C., which primarily contains the cyclomethicone tetramer (i.e. n=4), Dow Corning® 344 fluid having a viscosity of 2.5 centistokes and a boiling point of 178° C., which primarily contains the cyclomethicone pentamer (i.e. n=5), Dow Corning® 245 fluid having a viscosity of 4.2 centistokes and a boiling point of 205° C., which primarily contains a mixture of the cyclomethicone tetramer and pentamer (i.e. n=4 and 5), and Dow Corning® 345 fluid having a viscosity of 4.5 centistokes and a boiling point of 217°, which primarily contains a mixture of the cyclomethicone tetramer, pentamer, and hexamer (i.e. n=4, 5, and 6).

Dimethiconols are also suitable for use in the composition. These compounds can be represented by the chemical formulas R₃ SiO[R₂ SiO]ₓ SiR₂ OH and HOR₂ SiO[R₂ SiO]ₓ SiR₂ OH wherein R is an alkyl group (preferably R is methyl or ethyl, more preferably methyl) and x is an integer from 0 to about 500, chosen to achieve the desired molecular weight. Commercially available dimethiconols are typically sold as mixtures with dimethicone or cyclomethicone (e.g. Dow Corning® 1401, 1402, and 1403 fluids).

Polyalkylaryl siloxanes are also suitable for use in the composition. Polymethylphenyl siloxanes having viscosities from about 15 to about 65 centistokes at 25° C. are especially useful.

Preferred for use herein are organopolysiloxanes selected from the group consisting of polyalkylsiloxanes, alkyl substituted dimethicones, cyclomethicones, trimethylsiloxysilicates, dimethiconols, polyalkylaryl siloxanes, and mixtures thereof. More preferred for use herein are polyalkylsiloxanes and cyclomethicones. Preferred among the polyalkylsiloxanes are dimethicones.

(10) Vegetable oils and hydrogenated vegetable oils. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.

(11) Animal fats and oils (e.g., lanolin and derivatives thereof, cod liver oil).

(12) Fluorine-containing hydrocarbon fluids. Examples include but are not limited to, hydrofluoroethers from 3M Corporation, and perfluoropolyethers (Fomblin series manufactured by Montefluos, Demnum series manufactured by Daikin Industries and Krytox series manufactured by DuPont Corporation).

(13) Also useful are C4-C20 alkyl ethers of polypropylene glycols, C1-C20 carboxylic acid esters of polypropylene glycols, and di-C8-C30 alkyl ethers. Nonlimiting examples of these materials include PPG-14 butyl ether, PPG-15 stearyl ether, dioctyl ether, dodecyl octyl ether, and mixtures thereof.

2. Hydrophilic Polar Materials

Examples of hydrophilic polar materials include but are not limited to water, ethyl alcohol, isopropyl alcohol or any alcohol or glycol with a vapor pressure of about least about 0 mmHg to about 10 mm Hg at 1 atm and 25° C.

Examples of hydrophilic polar materials useful herein include, but are not limited to materials such as urea; guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol, and the like; polyethylene glycol; sugars and starches; sugar and starch derivatives (e.g., alkoxylated glucose); hyaluronic acid; chitin, starch-grafted sodium polyacrylates such as Sanwet (RTM) IM-1000, IM-1500, and IM-2500 (available from Celanese Superabsorbent Materials); lactamide monoethanolamine; acetamide monoethanolamine; propoxylated glycerol (as described in U.S. Pat. No. 4,976,953); and mixtures thereof.

B. Emulsifiers and Amphiphilic Molecules

The first composition of the present invention comprises an emulsifier, an amphiphilic molecule, or mixtures thereof. In a preferred embodiment, the composition contains from about 0.05% to about 10% emulsifier, more preferably from about 0.1% to about 7.5%, and even more preferably from about 0.5% to about 5%, by weight of the composition formed, of an emulsifier or amphiphilic molecule.

Known or conventional emulsifying agents can be used in the composition, provided that the selected emulsifying agent or amphiphilic molecule is miscible, soluble, or dispersable in the first composition and has the ability to: 1) effectively lower the interfacial tension between the external phases of the first and second compositions and/or to 2) emulsify the continuous phase of the second composition into the continuous phase of the first composition. Suitable emulsifiers can include any of a wide variety of nonionic, cationic, anionic, and zwitterionic emulsifiers disclosed in the prior patents and other references. See McCutcheon's, Detergents and Emulsifiers, North American Edition (1986), published by Allured Publishing Corporation; U.S. Pat. No. 5,011,681; U.S. Pat. No. 4,421,769; and U.S. Pat. No. 3,755,560.

Suitable emulsifier types include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, and mixtures thereof. Nonlimiting examples of suitable non-silicone-containing emulsifiers for use herein include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene-4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

Suitable emulsifiers may also include silicone emulsifiers, which are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

The dimethicone copolyol emulsifiers useful herein can be described by the following general structure:

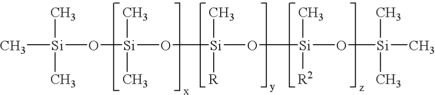

wherein R is C1-C30 straight, branched, or cyclic alkyl and R² is selected from the group consisting of

—(CH₂)ₙ—O—(CH₂CHR³O)ₘ—H,

and

—(CH₂)ₙ—O—(CH₂CHR³O)ₘ—(CH₂CHR⁴O)ₒ—H,

wherein n is an integer of from about 3 to about 10; R³ and R⁴ are selected from the group consisting of H and C1-C6 straight or branched chain alkyl such that R³ and R⁴ are not simultaneously the same; and m, o, x, and y are selected such that the molecule has an overall molecular weight from about 200 to about 10,000,000, with m, o, x, and y being independently selected from integers of zero or greater such that m and o are not both simultaneously zero, and z being independently selected from integers of 1 or greater. It is recognized that positional isomers of these copolyols can be achieved. The chemical representations depicted above for the R² moieties containing the R³ and R⁴ groups are not meant to be limiting but are shown as such for convenience.

Also useful herein, although not strictly classified as dimethicone copolyols, are silicone surfactants as depicted in the structures in the previous paragraph wherein R² is:

—(CH₂)ₙ—O—R⁵,

wherein R⁵ is a cationic, anionic, amphoteric, or zwitterionic moiety.

Nonlimiting examples of dimethicone copolyols and other silicone surfactants useful as emulsifiers herein include polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene)(propylene)oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples include the SILWET series and SILSOFT series available from Crompton/OSi. Other nonlimiting examples of dimethicone copolyols also include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993.

### SECOND COMPOSITION

The second composition of the cosmetic system comprises a continuous phase fluid, solid, gel, or semi-solid having an interfacial tension with the low volatility material from the first composition which is greater than 0 dyne/cm. The second composition is topically applied to the skin after the first composition.

By having an interfacial tension between the low-volatility materials of the continuous phase of the first composition (excluding any emulsifiers or amphiphilic molecules) and the continuous phase of the second composition greater than 0 dyne/cm, the two compositions are by definition at least partially immiscible. This means that the continuous phase fluids of the two compositions, when combined in a beaker at 1 atm and 25° C., will exhibit a meniscus. If there is a visible meniscus, the fluids are at least partially immiscible and therefore fit the description of the present invention. A method for determining if there is at least partial immiscibility between the compositions is described in the Handbook of Chemistry and Physics, 65^{th} Edition, from CRC Press, page C-703, titled “Miscibility of Organic Solvent Pairs,” by W. M. Jackson and J. S. Drury. Under circumstances where the low-volatility materials from the first composition are matched in refractive index to the materials in the continuous phase of the second composition, a meniscus may not be visible, though present. To account for this possibility, it may be necessary to dissolve a polar dye in the hydrophilic continuous phase fluid before mixing the 1^{st} and 2^{nd} composition fluid phases. The existence of at least partial immiscibility will be visually seen through the observation of a color intensity differentiation with the most intense color in the more polar phase where the greater presence of the polar dye molecules reside. Additionally, a tensiometer may be required to measure whether there exists a measurable interfacial tension between the two phases. An example of an acceptable method for measuring the interfacial tension is ASTM method D971-99a.

Suitable continuous phase fluids, solids, gels, or semi-solids for inclusion in the claimed cosmetic systems include, but are not limited to hydrophobic non-polar materials, hydrophilic polar materials, and mixtures thereof as described above in the first composition. The present cosmetic systems comprise from about 5.0% to about 90.0%, by weight of the cosmetic system of the continuous phase fluids, solids, gels, or semi-solids preferably from about 8.0% to about 70.0% by weight of the cosmetic system. In preferred embodiments, continuous phase fluids, solids, gels, or semi-solids are present in the cosmetic system in an amount of from about 10.0% to about 60.0%, by weight of the cosmetic system.

The second composition is not limited by its volatility or vapor pressure. Examples of suitable hydrophilic and hydrophobic fluids for the second composition are the same as those described in the first composition.

### OPTIONAL INGREDIENTS

A. Colorants

The compositions of the cosmetic system may further comprise a colorant. Suitable colorants include, but are not limited to, D&C Yellow No. 7, D&C Red No. 36, FD&C Red No. 4, D&C Orange No. 4, D&C Red No. 6, D&C Red No. 34, FD&C Yellow No. 6, D&C Red No. 33, FD&C Yellow No. 5, D&C Brown No. 1, D&C Red No. 17, FD&C Green No. 3, D&C Blue No. 4, D&C Yellow No. 8, D&C Orange No. 5, D&C Red No. 22, D&C Red No. 21, D&C Red No. 28, D&C Orange No. 11, D&C Yellow No. 10, D&C Violet No. 2, Ext. D&C Violet No. 2, D&C Green No. 6, D&C Green No. 5, D&C Red No. 30, D&C Green No. 8, D&C Red No. 7, FD&C Blue No. 1, D&C Yellow No. 7, D&C Red No. 27, D&C Orange No. 10, D&C Red No. 31, FD&C Red No. 40, D&C Yellow No. 11, Annatto extract, β carotene, guanine, carmine, aluminum powder, ultramarines, bismuth oxychloride, chromium oxide green, chromium hydroxide green, iron oxides, ferric ferrocyanide, manganese violet, titanium dioxide, titanated mica (i.e., mica coated with titanium dioxide), iron oxide titanated mica, zinc oxide, caramel coloring, mica, ferric ammonium ferrocyanide, dihydroxyacetone, guaiazulene, pyrophyllite, bronze powder, copper powder, aluminum stearate, calcium stearate, lactofavin, magnesium stearate, zinc stearate, capsanthin/capsorubin, bentonite, barium sulfate, calcium carbonate, calcium sulfate, carbon black, magnesium carbonate, magnesium silicate, colored silica, silica (including spherical silica, hydrated silica and silica beads), CI 10020, CI 11680, CI 15630, CI 15865, CI 16185, CI 16255, CI 16255, CI 45430, CI 69825, CI 73000, CI 73015, CI 74160, CI 75100, CI 77002, CI 77346, CI 77480, nylon powder, polyethylene powder, ethylene acrylates copolymer powder, methacrylate powder, polystyrene powder, silk powder, crystalline cellulose, starch, bismuth oxychloride, guanine, kaolin, chalk, diatomaceous earth, microsponges, boron nitride and the like. Additionally, lakes or composites of these colorants may also be used. Additional colorants, pigments, and powders useful herein are described in U.S. Pat. No. 5,505,937.

B. Film-Forming Agent

The compositions of the cosmetic system may further comprise a film-forming agent. Preferably, the compositions comprise from about 0% to about 20%, more preferably, from about 0.05% to about 10%, and even more preferably from about 0.1% to about 5%, by weight of the composition, of the film-forming agent.

Examples of suitable film forming agents useful in the compositions of the present kit include:

a) sulfopolyester resins, such as AQ sulfopolyester resins, such as AQ29D, AQ35S, AQ38D, AQ38S, AQ48S, and AQ55S (available from Eastman Chemicals);

b) polyvinylacetate/polyvinyl alcohol polymers, such as Vinex resins available from Air Products, including Vinex 2034, Vinex 2144, and Vinex 2019;

c) acrylic resins, including water dispersible acrylic resins available from National Starch under the trade name “Dermacryl”, including Dermacryl LT;

d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed., Vol. 17, pp. 198-257;

e) high molecular weight silicones such as dimethicone and organic-substituted dimethicones, especially those with viscosities of greater than about 50,000 mPas;

f) high molecular weight hydrocarbon polymers with viscosities of greater than about 50,000 mPas;

g) silicone-acrylate copolymers, including VS-70 (3M), SA-70 (3M), KP-545 (Shin-Etsu)

h) organosiloxanes, including organosiloxane resins, fluid diorganopolysiloxane polymers and silicone ester waxes;

i) polyurethanes, including Polyderm series of polymers from Alzo, Corp.; and

j) hydrophobic acrylate copolymers, including the acrylate/alkylmethacrylate copolymer Lipacryl (Rohm & Haas) or its emulsified, water dispersible version Allianz OPT (ISP).

Examples of these polymers and cosmetic compositions containing them are found in PCT publication Nos. WO96/33689, WO97/17058; and U.S. Pat. No. 5,505,937. Additional film forming polymers suitable for use herein include the water-insoluble polymer materials in aqueous emulsion and water soluble film forming polymers described in PCT publication No. WO98/18431. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Suitable film forming polymers also include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO₍₄₋ₙ)/2 where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrafunctional or SiO₂ “Q” units, otherwise known as “MQ” resins as disclosed in U.S. Pat. No. 5,330,747. In the present invention the ratio of the “M” to “Q” functional units is preferably about 0.7 and the value of n is 1.2. Organosiloxane resins such as these are commercially available such as Wacker 803 and 804 available from Wacker Silicones Corporation, and G. E. 1170-002 from the General Electric Company.

Other materials for enhancing wear or transfer resistance include trimethylated silica. Suitable silicas of this type and cosmetic compositions containing them are described in U.S. Pat. No. 5,800,816.

C. Absorbents

The compositions of the present invention may comprise one or more absorbent materials. These absorbents are useful for achieving the uptake of various fluids that are commonly present on the skin, e.g., perspiration, oil, and/or sebum. Suitable absorbents include, but are not limited to, silicas, silicates, polyacrylates, cross-linked silicones, cross-linked hydrocarbons, activated carbon, starch-based materials (for example cornstarch (topical starch), talc, rice starch, oat starch, tapioca starch, potato starch, legume starches, soy starch, turnip starch), microcrystalline cellulose (for example Avicel®), aluminum starch octenyl succinate (sold by National Starch & Chemical Co. as Dry Flo® Pure, Dry Flo® XT, Dry Flo® PC, and/or Dry Flo® AF (aluminum free grade)), kaolin, calcium silicate, amorphous silicas, calcium carbonate, magnesium carbonate, or zinc carbonate, and mixtures thereof. Some specific examples of the silicates and carbonates useful in the present invention are more fully explained in Van Nostrand Reinhold's Encyclopedia of Chemistry. 4th Ed. pages 155, 169,556, and 849, (1984).

D. Anti-Acne Actives

Examples of useful anti-acne actives of the present invention include, but are not limited to, the keratolytics such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate, and cholate.

E. Antiperspirant Actives

Antiperspirant actives may also be included in the compositions of the present invention. Suitable antiperspirant actives include astringent metallic salts, especially the inorganic and organic salts of aluminum zirconium and zinc, as well as mixtures thereof. Particularly preferred are the aluminum containing and/or zirconium-containing materials or salts, such as aluminum halides, aluminum chlorohydrate, aluminum hydroxyhalides, zirconyl oxyhalides, zirconyl hydroxyhalides, and mixtures thereof.

F. Anti-Wrinkle and Anti-Skin Atrophy Actives

Examples of anti-wrinkle and anti-skin atrophy actives useful in the present invention include, but are not limited to, retinoic acid and its derivatives (e.g., cis and trans); retinol; retinyl esters; niacinamide, and derivatives thereof; sulfur-containing D and L amino acids and their derivatives and salts, particularly the N-acetyl derivatives, a preferred example of which is N-acetyl-L-cysteine; thiols (e.g., ethane thiol); terpene alcohols (e.g., farnesol); hydroxy acids, phytic acid, lipoic acid; lysophosphatidic acid, alpha-hydroxy acids (e.g., lactic acid and glycolic acid), beta-hydroxy acids (e.g., salicylic acid), and skin peel agents (e.g., phenol and the like).

G. Artificial Tanning Actives and Accelerators

Examples of artificial tanning actives and accelerators useful in the compositions of the present invention include, but are not limited to, dihydroxyacetaone, tyrosine, tyrosine esters such as ethyl tyrosinate, phospho-DOPA, and mixtures thereof.

H. Astringents

The compositions of the present invention may include astringents. Astringents are useful for shrinking pores of the skin. Suitable astringents include, but are not limited to, clove oil, fomes officinalis extract, spiraea ulmaria extract, menthol, camphor, eucalyptus oil, eugenol, menthyl lactate, witch hazel distillate, aluminum salts, tannins, ethanol, and combinations thereof.

I. Hydrophilic Conditioning Agents

The present invention can also comprise or more hydrophilic conditioning agents. Nonlimiting examples of hydrophilic conditioning agents include those selected from the group consisting of polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof. Specific examples of useful hydrophilic conditioning agents include materials such as urea; guanidine; glycolic acid and glycolate salts (e.g., ammonium and quaternary alkyl ammonium); lactic acid and lactate salts (e.g., ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, polypropylene glycols such as PPG-9, PPG-12, PPG-15, PPG-17, PPG-20, PPG-26, PPG-30, PPG-34; alkoxylated glucose; hyaluronic acid; cationic skin conditioning polymers (e.g., quaternary ammonium polymers such as Polyquaternium polymers); and mixtures thereof. Glycerol, in particular, is a preferred hydrophilic conditioning agent in the present invention. Also useful are materials such as aloe vera in a variety of forms (e.g., aloe vera gel), chitosan and chitosan derivatives (e.g., chitosan lactate, lactamide monoethanolamine); acetamide monoethanolamine; and mixtures thereof. Also useful are propoxylated glycerols as described in propoxylated glycerols described in U.S. Pat. No.4,976,953.

J. Hydrophobic Conditioning Agents

The composition may comprise one or more hydrophobic conditioning agents. Preferred hydrophobic conditioning agents are selected from the group consisting of mineral oil, petrolatum, lecithin, hydrogenated lecithin, lanolin, lanolin derivatives, C7-C40 branched chain hydrocarbons, C1-C30 alcohol esters of Cl-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkylarylsiloxanes, cylcomethicones having 3 to 9 silicone atoms, vegetable oils, hydrogenated vegetable oils, polypropylene glycol C4-C20 alkyl ethers, di C8-C30 alkyl ethers, and combinations thereof.

K. Light Diffusers

The compositions may comprise a light diffuser. Light diffusers are useful for improving skin appearance by minimizing the appearance of texture such as pores and fine lines. Suitable light diffusers for inclusion into the compositions of the present system include, but are not limited to silica, nylon, polyethylene, polymethyl methacrylate, polystyrene, methylsiloxane copolymer, polytetrafluoroethylene copolymer, boron nitride, silicone resin powders, silicone rubber powders, ethylene acrylate copolymers, mica, titanium dioxide, iron oxides, zinc oxide, and combinations thereof.

L. Oil-soluble Polymeric Gelling Agents

The compositions of the present invention may optionally comprise one or more polymeric materials that are oil-soluble and form a gel with hydrophobic materials (e.g., oils) that are contained in the compositions. Such polymers are beneficial for structuring these materials resulting in flexible gels with improved stability and shear-resistance.

Particularly suitable are at least partially cross-linked oil-soluble polymeric materials with a softening point <160° C. Suitable materials come from the chemical groups of PE (polyethylenes), PVA (polyvinyl alcohols) and derivatives, PVP (polyvinylpyrrolidones) and derivatives, PVP/Alkene Copolymers, PVP/VA copolymers, PVM/MA (methyl vinyl ether/maleic anhydride) copolymers and their esters and ethers, particularly poly (alkyl vinyl ether-co-maleic anhydride) copolymers, ethylene/VA copolymers, acrylates/alkyl methacrylate copolymer, styrene/isoprene, styrene/ethylene/butylene, styrene/ethylene/propylene, styrene/ethylene/butylene/styrene, styrene/butadiene copolymers, benotnite clays, hectorite clays, organix waxes and silicone waxes. Suitable materials are available e.g. from Dupont (ELVAX® types), BASF (LUVISKOL® types), Shell (KRATON® polymers), ISP (PVP, GANTREZ®, GANEX® and ALLIANZ OPT® types) and Rohm & Haas (LIPACRYL®).

M. Hydrophilic Gelling Agent

The compositions of the invention may optionally contain a hydrophilic gelling agent. The gelling agent preferably has a viscosity (1% aqueous solution, 20° C., Brookfield RVT) of at least about 4000 mPas, more preferably at least about 10,000 mPas and even more preferably at least 50,000 mPas.

Suitable hydrophilic gelling agents can generally be described as water-soluble or colloidally water-soluble polymers, and include cellulose ethers (e.g. hydroxyethyl cellulose, methyl cellulose, hydroxypropylmethyl cellulose), bentonite clays, hectorite clays, polyvinylpyrrolidone, polyvinylalcohol, polyquaternium-10, guar gum, hydroxypropyl guar gum, and xanthan gum.

Among suitable hydrophilic gelling agents are acrylic acid/ethyl acrylate copolymers and the carboxyvinyl polymers sold by the B. F. Goodrich Company under the trademark of Carbopol resins. These resins consist essentially of a colloidally water-soluble polyalkenyl polyether crosslinked polymer of acrylic acid crosslinked with from about 0.75% to about 2.00% of a crosslinking agent such as for example polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule. Also suitable for use herein are hydrophobically-modified cross-linked polymers of acrylic acid having amphipathic properties available under the Trade Name Carbopol 1382, Carbopol 1342 and Pemulen TR-1 (CTFA Designation: Acrylates/10-30 Alkyl Acrylate Crosspolymer). A combination of the polyalkenyl polyether cross-linked acrylic acid polymer and the hydrophobically modified cross-linked acrylic acid polymer is also suitable for use herein. Other suitable gelling agents suitable for use herein are oleogels such as trihydroxystearin and aluminium magnesium hydroxy stearate. The gelling agents herein are particularly valuable for providing excellent stability characteristics over both normal and elevated temperatures.

Neutralizing agents suitable for use in neutralizing acidic group containing hydrophilic gelling agents herein include sodium hydroxide, potassium hydroxide, ammonium hydroxide, monoethanolamine, diethanolamine and triethanolamine.

N. Crosslinked Silicone Polymers

The composition of the present invention may optionally include a polymer that is non-linear in nature. Suitable polymers for inclusion in the claimed compositions include, but are not limited to polysiloxanes that are crosslinked organopolysiloxane polymer gel networks. For instance, particularly well-suited crosslinked organopolysiloxane polymer gel networks are formed from polymerization of an epoxy functional organosiloxane in the presence of an acid catalyst. The organopolysiloxane polymer is a crosslinked organopolysiloxane polymer gel network selected from non-emulsifying polymer gel networks, emulsifying polymer gel networks, and combinations thereof. Specific examples of such are described in U.S. Pat. No. 6,531,540 B1, U.S. Pat. No. 6,538,061 B2, U.S. Pat. No. 6,444,745 B1, U.S. Pat. No. 6,346,583 B1, U.S. Pat. No. 5,654,362, U.S. Pat. No. 5,811,487, U.S. Pat. No. 5,880,210, U.S. Pat. No. 5,889,108, U.S. Pat. No. 5,929,164, U.S. Pat. No. 5,948,855, U.S. Pat. No. 5,969,035, U.S. Pat. No. 5,977,280, U.S. Pat. No. 6,080,394, U.S. Pat. No. 6,168,782, U.S. Pat. No. 6,177,071, U.S. Pat. No. 6,200,581, U.S. Pat. No. 6,207,717, U.S. Pat. No. 6,221,927, U.S. Pat. No. 6,221,979, U.S. Pat. No. 6,238,657, and U.S. Pat. No. 4,987,169.

Suitable organopolysiloxane polymer network powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200, and hybrid silicone powders that contain a phenyl group such as Shin-Etsu's KSP-300; and Dow Corning's DC 9506.

Preferred organopolysiloxane compositions are dimethicone/vinyl dimethicone crosspolymers. Such dimethicone/vinyl dimethicone crosspolymers are supplied by a variety of suppliers including Dow Corning (DC 9040 and DC 9041), General Electric (Velvesil 125), General Electric (SFE 839), Shin Etsu (KSG-15, 16, 18 [dimethicone/phenyl vinyl dimethicone crosspolymer] and KSG-21 [dimethicone copolyol crosspolymer]), Grant Industries (Gransil™ line of materials), lauryl dimethicone/vinyl dimethicone crosspolymers supplied by Shin Etsu (e.g., KSG-41, KSG-42, KSG-43, and KSG-44), lauryl dimethicone/dimethicone copolyol crosspolymers also supplied by Shin-Etsu (e.g., KSG-31, KSG-32, KSG-33, and KSG-34), and Wacker (Belsil RG-100) Additional polymers from Shin-Etsu which are suitable for use in the present invention include KSG-210, -310, -320, -330, and -340. Crosslinked organopolysiloxane polymer gel networks useful in the present invention and processes for making them are further described in U.S. Pat. No. 4,970,252; U.S. Pat. No. 5,760,116; U.S. Pat. No. 5,654,362; and Japanese Patent Application JP 61-18708.

O. Sunscreen Actives

Also useful herein are sunscreen actives. A wide variety of sunscreen actives are described in U.S. Pat. No. 5,087,445; U.S. Pat. No. 5,073,372; U.S. Pat. No. 5,073,371; and Sagarin, et al., at Chapter VIII, pages 189 et seq., of Cosmetics Science and Technology. Nonlimiting examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991. Especially preferred examples of these sunscreens include those selected from the group consisting of 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof. Exact amounts of sunscreens that can be employed will vary depending upon the sunscreen chosen and the desired Sun Protection Factor (SPF) to be achieved. SPF is a commonly used measure of photoprotection of a sunscreen against erythema.

P. Additional Optional Ingredients

The compositions of the present invention may also include ingredients classified as desquamating agents, skin lightening agents, skin soothing and skin healing actives, vitamin compounds and precursors, chelators, enzymes, flavinoids (broadly disclosed in U.S. Pat. Nos. 5,686,082 and 5,686,367), and sterol compounds.

### ASSOCIATED METHODS

Applicants have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appearance of spider vessels and varicose veins; 11) methods of masking the appearance of vellus hair on skin; 12) methods of concealing blemishes and/or imperfections in human skin, including acne, age spots, freckles, moles, scars, under eye circles, birth marks, post-inflammatory hyperpigmentation; 13) methods of enhancing or modifying skin color such as lightening, darkening, making more pink, making more yellow, making less dull, making less ashy, making less orange, making more radiant; 14) methods of artificial tanning; 15) methods of concealing vitiligo; 16) methods of concealing damage incurred to the skin as a result of trauma, e.g., cosmetic surgery, burns, stretching of skin, etc.; and 17) methods of concealing wrinkles, fine lines, pores, uneven skin surfaces, etc. Each of the methods discussed herein involve topical application of the claimed compositions to skin.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples 1-5

Water in Silicone and Water in Oil First Composition Examples:

| | Ingredient | Example 1 | Example 2 | Example 3 | Example 4 | Example 5 |
|---|---|---|---|---|---|---|
| A1 | Dodecamethyl | 12.450 | 12.000 | 11.550 | 12.000 | — |
| | cyclohexasiloxane | | | | | |
| A2 | Silica¹ | 2.000 | 2.000 | 2.000 | 2.000 | 2.000 |
| A3 | Tridecylneopentanoate | 5.000 | 6.000 | 5.000 | 6.000 | 10.000 |
| A4 | polyethylene glycol 20 | — | — | — | — | 2.000 |
| | sorbitan monolaurate | | | | | |
| A5 | Isododecane | — | — | — | — | 18.000 |
| A6 | Isostearyl Isostearate | — | — | 3.000 | — | 12.000 |
| A7 | Dimethicone (500 cSt) | — | — | — | 30.00 | |
| A8 | Cyclopentasiloxane (and) | 8.000 | 8.900 | — | 8.900 | — |
| | dimethicone copolyol | | | | | |
| A9 | Cyclopentasiloxane (and) | 22.000 | 30.000 | 24.000 | | — |
| | C30-45 Alkyl Cetearyl | | | | | |
| | Dimethicone Crosspolymer | | | | | |
| | (87.5%)² | | | | | |
| A10 | Cyclopentasiloxane (and) | 9.500 | — | — | — | — |
| | C30-45 Alkyl Cetearyl | | | | | |
| | Dimethicone Crosspolymer | | | | | |
| | (70.0%)² | | | | | |
| A11 | Cyclopentasiloxane (and) | — | — | 12.000 | — | — |
| | C30-45 Alkyl Cetearyl | | | | | |
| | Dimethicone Crosspolymer | | | | | |
| | (and) PEG/PPG 20/15 | | | | | |
| | Dimethicone³ | | | | | |
| B1 | Zinc Oxide | 3.000 | 3.00 | 3.000 | 3.00 | 3.000 |
| C1 | Propylparaben | 0.200 | 0.200 | 0.200 | 0.200 | 0.200 |
| C2 | Octyl methoxycinnimate | 6.000 | 6.000 | 6.000 | 6.000 | 6.000 |
| D1 | SD alcohol | 8.500 | 8.500 | 10.000 | 8.500 | — |
| D2 | Deionized water | 16.050 | 16.500 | 15.950 | 16.500 | 40.5 |
| D3 | Sodium chloride | 1.000 | 1.000 | — | 1.000 | — |
| D4 | Methylparaben | 0.200 | 0.200 | 0.200 | 0.200 | 0.200 |
| D5 | Glycerin | 4.000 | 4.000 | 5.000 | 4.000 | 4.000 |
| D6 | Trisodium edetate | 0.100 | 0.100 | 0.100 | 0.100 | 0.100 |
| D7 | Phemoxyethanol | 0.400 | — | 0.400 | — | 0.400 |
| E1 | Acrylates/C12-22 | 1.600 | 1.600 | 1.600 | 1.600 | 1.600 |
| | Alkylmethacrylate⁴ | | | | | |

¹Kobo Products - Silica Shells
²General Electric Silicones - Velvesil 125
³General Electric Silicones - 1120-21-381
⁴Rohm and Haas - Allianz OPT

Steps:

1. Combine ingredients D1 and D5 into large stainless steel breaker. Provide maximum prop mixer blending without air incorporation.

2. Add Phase A and B ingredients to a stainless steel vessel. Mix mix at high shear for 15 minutes or until uniformity is achieved.

3. Add Phase C ingredients into a separate beaker. Manually mix until paraben is dissolved.

4. Add Phase C to batch. Continue high shear until homogenous.

5. Once Phase ABC are uniform insert homogenizer then add Phase D and continue homogenization until uniformity is achieved.

6. Add Phase E and blend with prop mixer until uniform in appearance and consistency.

### Examples 6-10

Silicone in Water and Oil in Water Second Composition Examples:

| | Ingredient | Example 6 | Example 7 | Example 8 | Example 9 | Example 10 |
|---|---|---|---|---|---|---|
| A1 | Decamethylcyclopentasiloxane | 9.000 | 9.145 | 9.145 | — | — |
| A2 | Dodecamethyl | 2.000 | 2.065 | 5.065 | — | — |
| | cyclohexasiloxane | | | | | |
| A3 | Tridecyl Neopentanoate | 8.000 | 8.000 | 10.000 | 12.000 | 12.00 |
| A4 | PCA Dimethicone | 2.000 | 2.000 | 2.000 | — | — |
| A5 | Isododecane | — | — | — | 20.000 | 20.000 |
| A6 | Propylparaben | 0.200 | 0.150 | 0.150 | .150 | .150 |
| A7 | Arachadyl Behenate | — | 0.300 | 0.300 | 0.300 | 0.300 |
| A8 | Stearyl Alcohol | 0.625 | 0.750 | 0.750 | 0.7500 | 0.7500 |
| A9 | Behenyl Alcohol | 0.625 | — | — | — | — |
| A10 | Methyl Glucose | 1.200 | — | — | — | — |
| | Sesquistearate | | | | | |
| B1 | Titanium Dioxide (And) | 9.075 | 9.075 | — | 9.075 | — |
| | Polyglyceryl-4 Isostearate | | | | | |
| | (And) Cetyl Dimethicone | | | | | |
| | Copolyol (And) Hexyl | | | | | |
| | Laurate (And) Isopropyl | | | | | |
| | Titanium Triisostearate¹ | | | | | |
| B2 | Iron Oxide (CI 77492) | 0.811 | 0.811 | — | 0.811 | — |
| | (And) Polyglyceryl-4 | | | | | |
| | Isostearate (And) Cetyl | | | | | |
| | Dimethicone Copolyol | | | | | |
| | (And) Hexyl Laurate | | | | | |
| | (And) Isopropyl Titanium | | | | | |
| | Triisostearate¹ | | | | | |
| B3 | Iron Oxide (CI 77491) | 0.262 | 0.262 | — | 0.262 | — |
| | (And) Polyglyceryl-4 | | | | | |
| | Isostearate (And) Cetyl | | | | | |
| | Dimethicone Copolyol | | | | | |
| | (And) Hexyl Laurate | | | | | |
| | (And) Isopropyl Titanium | | | | | |
| | Triisostearate¹ | | | | | |
| B4 | Iron Oxide (CI 77499) | 0.143 | 0.143 | — | 0.143 | — |
| | (And) Polyglyceryl-4 | | | | | |
| | Isostearate (And) Cetyl | | | | | |
| | Dimethicone Copolyol | | | | | |
| | (And) Hexyl Laurate | | | | | |
| | (And) Isopropyl Titanium | | | | | |
| | Triisostearate¹ | | | | | |
| B5 | Silica² | — | 2.00 | — | 2.000 | 2.000 |
| C1 | Deionized Water | 53.655 | 52.000 | 59.291 | 40.579 | 51.500 |
| C2 | Methylparaben | 0.250 | 0.200 | 0.200 | 0.200 | 0.200 |
| C3 | 2-Phenoxyethanol | — | 0.500 | 0.500 | 0.500 | 0.500 |
| C4 | Hydroxypropyl Starch | — | 2.000 | 2.000 | 2.000 | 2.000 |
| | Phosphate | | | | | |
| C5 | Glycerin | 2.250 | 2.250 | 2.250 | 2.250 | 2.250 |
| C6 | Butylene Glycol | 2.250 | 2.250 | 2.250 | 2.250 | 2.250 |
| C7 | Polyvinylpyrrolidone | — | 1.000 | 1.000 | 1.000 | 1.000 |
| C8 | Trisodium Edetate | 0.100 | 0.100 | 0.100 | 0.100 | 0.100 |
| C9 | Sucrose Palmitate (and) | — | 2.000 | 2.000 | 2.000 | 2.000 |
| | Glyceryl Stearate (and) | | | | | |
| | Glyceryl Stearate Citrate | | | | | |
| | (and) Sucrose (and) | | | | | |
| | Mannan (and) Xanthan | | | | | |
| | Gum³ | | | | | |
| C10 | Aluminum Starch | 1.004 | 3.00 | 3.000 | 3.000 | 3.000 |
| | Octenylsuccinate⁴ | | | | | |
| C11 | POE 20 Methyl Glucose | 1.800 | | | | |
| | Sesquistearate | | | | | |
| D1 | 2-Phenoxyethanol | 0.750 | | | | |
| D2 | Hydroxyethyl | 4.000 | | | | |
| | Acrylate/Sodium | | | | | |
| | Acryloyldimethyl Taurate | | | | | |
| | Copolymer and Squalane | | | | | |
| | and Polysorbate 60⁵ | | | | | |

¹Kobo Products - ITT Coated Pigments
²Kobo Products - Silica Shells
³Uniquima - Arlatone V-175
⁴National Starch - Dry Flo Elite BN
⁵Seppic - Simulgel NS

Steps for Example 6:

1. Combine phase C ingredients and propeller mix for 10 minutes. Heat phase C to 75° C.

2. In separate stainless steel vessel, combine phase A and B ingredients and use high shear mixing to disperse particles (6000 rpm) for 20-30 minutes. During the mixing phase, heat to 75° C.

3. Introduce Phase AB into Phase C while using high shear (6000 rpm). Homogenize for 20-30 minutes.

4. Cool product to below 60° C. and add D1. Shear for 10 minutes.

5. Add D2 and propeller mix for 10 minutes or until uniform.

Steps for Example 7-10:

1. Combine ingredients C1 and C9 with maximum propeller.

2. Add Phase C ingredients 2,3,5,6,7,8 and 10. Provide maximum prop mixer blending without air incorporation

3. Heat Phase C to 70-80° C. Once batch reaches 70-80° C. add 50% C4.

4. Add Phase A ingredients 1-5 to separate vessel and begin homogenizing batch.

5. Heat Phase A to 70-80° C.

6. Add Phase B to Phase A shear on HIGH for approximately 20-30 minutes.

7. Once Phase AB reaches 70-80° C. add Phase A ingredients 6-8.

8. Transfer Phase AB to Phase C while prop mixing. Blend until uniform in appearance.

9. Homogenize batch with high shear. Add remaining 50% C4. Maintain until uniformity is achieved.

### Examples 11-15

Oil in Water and Silicone in Water First Composition Examples:

| | | Example | Example | Example | Example | Example |
|---|---|---|---|---|---|---|
| | Ingredient | 11 | 12 | 13 | 14 | 15 |
| A1 | Decamethylcyclopentasiloxane | 9.135 | 9.135 | 9.135 | 9.135 | 9.135 |
| A2 | Dodecamethyl | 2.065 | 2.065 | 2.065 | 2.065 | 2.065 |
| | cyclohexasiloxane | | | | | |
| A3 | Tridecyl Neopentanoate | 5.000 | 5.000 | 5.000 | 5.000 | 5.000 |
| A4 | PCA Dimethicone | 2.000 | 2.000 | 2.000 | 2.000 | 2.000 |
| A5 | Propylparaben | 0.150 | 0.150 | 0.150 | 0.150 | 0.150 |
| A6 | Arachadyl behenate | 0.300 | 0.300 | 0.300 | 0.300 | 0.300 |
| A7 | Stearyl Alcohol | 0.750 | 0.750 | 0.750 | 0.750 | 0.750 |
| B1 | Silica¹ | 2.000 | 2.000 | 2.000 | 2.000 | 2.000 |
| B2 | Titanium Dioxide | — | 0.500 | 0.500 | 0.500 | 0.500 |
| B3 | Polymethylsilsequioxane² | — | 2.000 | 2.000 | 2.000 | — |
| B4 | Aluminum Starch | 1.300 | 1.300 | 1.300 | 1.300 | 1.300 |
| | Octenylsuccinate (and) | | | | | |
| | Boron Nitride³ | | | | | |
| C1 | Deionized Water | 57.000 | 49.000 | 49.000 | 54.000 | 41.000 |
| C2 | Methylparaben | 0.200 | 0.200 | 0.200 | 0.200 | 0.200 |
| C3 | Phenoxyethanol | 0.500 | 0.500 | 0.500 | 0.500 | 0.500 |
| C4 | Hydroxypropyl Starch | 2.000 | 2.000 | 2.000 | 2.000 | 2.000 |
| | Phosphate⁴ | | | | | |
| C5 | Glycerin | 2.250 | 4.000 | 10.000 | 10.000 | 10.000 |
| C6 | Butylene Glycol | 2.250 | 10.000 | 5.000 | 5.000 | 5.000 |
| C7 | Propylene Glycol | 10.00 | 6.000 | 5.000 | — | 15.000 |
| C8 | Polyvinylpyrrolidone | 1.000 | 1.000 | 1.000 | 1.000 | 1.000 |
| C9 | Trisodium Edetate | 0.100 | 0.100 | 0.100 | 0.100 | 0.100 |
| C10 | Sucrose Palmitate (and) | 2.000 | 2.000 | 2.000 | 2.000 | 2.000 |
| | Glyceryl Stearate (and) | | | | | |
| | Glyceryl Stearate Citrate | | | | | |
| | (and) Sucrose (and) Mannan | | | | | |
| | (and) Xanthan Gum⁵ | | | | | |

¹Kobo Products - Silica Shells
²General Electric - Tospearl 145
³National Starch - Dry Flow Elite BN
⁴National Starch - Structure Zea
⁵Uniquima - Arlatone V-175

Steps:

1. Combine ingredients C1 and C 10 with maximum propeller.

2. Add Phase C ingredients 2,3,5,6,7,8, and 9. Provide maximum prop mixer blending without air incorporation

3. Heat Phase C to 70-80° C. Once batch reaches 70-80° C. add C4.

4. Add Phase A ingredients 1-5 to separate vessel and begin homogenizing batch.

5. Heat Phase A to 70-80° C.

6. Add Phase B to Phase A shear on HIGH for approximately 20-30 minutes.

7. Once Phase AB reaches 70-80° C. add Phase A ingredients 6 -7.

8. Transfer Phase AB to Phase C while prop mixing. Blend until uniform in appearance.

9. Homogenize batch with high shear. Maintain until uniformity is achieved

### Examples 16-21

Water in Silicone and Water in Oil Second Composition Examples:

| | | Example | Example | Example | Example | Example | Example |
|---|---|---|---|---|---|---|---|
| | Ingredient | 16 | 17 | 18 | 19 | 20 | 21 |
| A1 | Cyclopetnacsiloxane and | 8.000 | 8.000 | — | — | 8.000 | 8.000 |
| | dimethicone coplolyol | | | | | | |
| A2 | polyethylene glycol 20 | — | — | 2.000 | 2.000 | — | — |
| | sorbitan monolaurate | | | | | | |
| A3 | Isostearyl Isostearate | | | 16.000 | 16.000 | | |
| A4 | Tridecyl neopentanoate | 6.000 | 6.000 | 6.000 | 6.000 | 6.000 | 6.000 |
| A5 | Cetyl Dimethicone | 2.000 | 2.000 | 2.000 | 2.000 | 2.000 | 2.000 |
| | Colpolyol¹ | | | | | | |
| A6 | Cyclohexasiloxane | — | — | — | — | | |
| A7 | Cyclopentasiloxane | 31.700 | 25.700 | 15.700 | 15.700 | 10.700 | 4.700 |
| B1 | Ethoxylated C10-16 | 0.500 | 0.500 | 0.500 | 0.500 | 0.500 | 0.500 |
| | Alcohols² | | | | | | |
| B2 | Propylparaben | 0.150 | 0.150 | 0.150 | 0.150 | 0.150 | 0.150 |
| C2 | Aluminum Starch | 3.500 | 3.500 | 3.500 | 3.500 | 3.500 | 3.500 |
| | Octenylsuccinate (and) | | | | | | |
| | Boron Nitride³ | | | | | | |
| C3 | Polymethylsilsequioxane⁴ | 1.500 | 1.500 | 1.500 | 1.500 | 1.500 | 1.500 |
| C4 | Hexamethylene | 1.500 | 1.500 | 1.500 | 1.500 | 1.500 | 1.500 |
| | Diisocyanate/Polypropylene/ | | | | | | |
| | Polycaprolactone | | | | | | |
| | Crosspolymer (AND) | | | | | | |
| | Silica⁵ | | | | | | |
| C5 | Dimethicone/vinyldimethicone | 1.500 | 1.500 | 1.500 | 1.500 | 1.500 | 1.500 |
| | crosspolymer⁶ | | | | | | |
| C6 | Cyclopentasiloxane | — | — | — | — | 20.000 | 20.000 |
| | (and) C30-45 Alkyl | | | | | | |
| | Cetearyl Dimethicone | | | | | | |
| | Crosspolymer⁷ | | | | | | |
| C7 | Red Iron Oxides | — | 0.800 | 0.800 | — | — | 0.800 |
| C8 | Yellow Iron Oxides | — | 1.000 | 1.000 | — | — | 1.000 |
| C9 | Black Iron Oxides | — | 0.200 | 0.200 | — | — | 0.200 |
| C10 | Titianium Dioxide | — | 8.000 | 8.000 | — | — | 8.000 |
| D1 | Deionized Water | 48.000 | 43.000 | 43.000 | 54.000 | 48.000 | 44.000 |
| D2 | Polyvinylpyrrolodone | — | 1.000 | 1.000 | — | 1.000 | 1.000 |
| D3 | Silica⁸ | 1.000 | 1.000 | 1.000 | 1.000 | 1.000 | 1.000 |
| D4 | Phenoxyethanol | 0.250 | 0.250 | 0.250 | 0.250 | 0.250 | 0.250 |
| D5 | Trisodium EDTA | 0.100 | 0.100 | 0.100 | 0.100 | 0.100 | 0.100 |
| D6 | Sodium Chlosride | 2.000 | 2.000 | 2.000 | 2.000 | 2.000 | 2.000 |
| D7 | Sodium Dehydroacetate | 0.300 | 0.300 | 0.300 | 0.300 | 0.300 | 0.300 |
| | Monohydrate | | | | | | |

¹DeGussa Goldsmith - Abil WE-09
²Rhodia - Rhodasurf L-7/90
³National Starch - Dry Flo Elite BN
⁴GE - Tospearl 145A
⁵Kobo - BPD-500T
⁶Dow Corning - 9506 Powder
⁷GE - Velvesil 125
⁸Kobo - Silica Shells

Steps:

1. Combine Deionized Water and Silica Shells of Phase D ingredients and mix with propeller or dispersator until homogeneity is achieved.

2. Add remaining Phase D ingredients and continue propeller or dispersator blending.

3. Combine Phase A ingredients in jacketed vessel and begin mixing with rotor stator mill. Recirculate cold water through jacketed vessel.

4. Blend Phase B ingredients together for ten minutes. Add blended Phase B into Phase A ingredients.

5. Add Phase C Ingredients into Phase AB. Shear Phase ABC until it is completely deagglomerated and pigments have been reduced to their primary particle size.

6. Emulsify Phase D into Phase ABC under moderate shear. Blend Phase ABCD with sweep wall mixing until uniform.

### Example 22

A powder foundation that is suitable for use as the second composition.

| Phase A | |
|---|---|
| Talc | 23.90 |
| Mica | 17.66 |
| Mica (sericite) | 29.04 |
| Titanium Dioxide | 11.60 |
| Nylon-12 | 1.76 |
| Silica | 2.64 |
| Propylparaben, NF | 0.10 |
| Methylparaben, NF | 0.30 |
| Sodium Dehydroacetate, NF | 0.10 |
| Red Iron Oxide | 0.43 |
| Black Iron Oxide | 0.29 |
| Yellow Iron Oxide | 0.50 |
| Phase B | |
| Dimethicone (and) | 6.43 |
| Trimethylsiloxysilicate | |
| Dioctyl Succinate | 0.80 |
| Octyl Hydroxystearate | 0.70 |
| Cholesterol Hydroxystearate | 1.05 |
| Tocopherol | 0.01 |
| Octyl Methoxycinnamate | 2.69 |
| | 100 |

Combine Phase A ingredients with bulk mixing (e.g. Ribbon Blender, Double cone blender or by hand) followed by high shear mixing (e.g. hammer mill, pulveriser or chopping blades) to break down any particle agglomerates. Combine Phase B ingredients with a propeller mixer and heat to 75° C. Add Phase B ingredients to phase A and disperse with a combination of bulk and high shear mixing.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A cosmetic system suitable for application as a multi-step skin cosmetic product, said system comprising:
a. a first composition that comprises:
i. from about 5 to about 90 weight percent of a low volatility material with a vapor pressure of at least about 0 mmHg to about 10 mmHg at 1 atm and 25° C.; and
ii. from about 0.05 to about 10 weight percent of an emulsifier or amphiphilic molecule which is soluble or dispersible in the said low-volatility material; and
b. a second composition that comprises a continuous phase fluid, solid, gel, or semi-solid having an interfacial tension with said low-volatility material from said first composition which is greater than 0 dyne/cm,
wherein said second composition is topically applied to skin after said first composition.

2. The cosmetic system of claim 1 wherein said first composition comprises from about 8 to about 70 weight percent of said low volatility material.

3. The cosmetic system of claim 1 wherein said first composition comprises from about 10 to about 60 weight percent of said low volatility material.

4. The cosmetic system of claim 1 wherein said first composition comprises from about 0.1 to about 7.5 weight percent of said emulsifier or amphiphilic molecule.

5. The cosmetic system of claim 1 wherein said first composition comprises from about 0.5 to about 5 weight percent of said emulsifier or amphiphilic molecule.

6. The cosmetic system of claim 1 wherein said first composition, said second composition, or both said first composition and said second composition further comprise a colorant.

7. The cosmetic system of claim 1 wherein said first composition, said second composition, or both said first composition and said second composition further comprise a film-forming agent.

8. The cosmetic system of claim 1 wherein said first composition, said second composition, or both said first composition and said second composition further comprise a crosslinked silicone polymer.

9. The cosmetic system of claim 8 wherein said crosslinked silicone polymer is present in said first composition.

10. The cosmetic system of claim 1 wherein said first composition, said second composition, or both said first and said second composition further comprise an absorbent material.

11. The cosmetic system of claim 1 wherein said cosmetic product is a foundation.

12. A method of providing a multi-step cosmetic product to skin, said method comprising the steps of:
a. applying a first composition to said skin that comprises:
i. from about 5 to about 90 weight percent of a low-volatility material with a vapor pressure of at least about 0 mmHg to about 10 mmHg at 1 atm and 25° C.; and
ii. from about 0.05 to about 10 weight percent of an emulsifier which is soluble or dispersible in the said low-volatility material; and
b. applying a second composition to said skin that comprises a continuous phase fluid, solid, gel, or semi-solid having an interfacial tension with said low-volatility material from said first composition which is greater than 0 dyne/cm.
